(19) United States
(12) Patent Application Publication
(10) Pub. No.: US 2005/0273154 A1
(43) Pub. Date: Dec. 8, 2005

(54) Bifurcated stent graft and apparatus for making same

(76) Inventor: William M. Colone, Phoenix, AZ (US)

Correspondence Address: SQUIRE, SANDERS & DEMPSEY L.L.P.;Two Renaissance Square, Suite 2700, 40 North Central Avenue, Phoenix, AZ, 85004-4498, US

(21) Appl. No.: 10/865,488

(22) Filed: Jun. 8, 2004

(51) Int. Cl. A61F 2/06

(52) U.S. Cl. 623/1.13; 623/1.35; 264/320; 156/184

(57) ABSTRACT

The present invention includes a tool for forming an intravascular device such as a bifurcated stent graft. The tool includes a plurality of tool pieces wherein at least one tool piece is removably attached to another tool piece, and an external surface for assisting in forming the intravascular device. The tool may be used to form a stent graft by applying a first layer of biocompatible material over an external surface of the tool, applying a support structure over the first layer, and applying a second layer of biocompatible material over the support structure and the first layer. The present invention also includes two-piece stent grafts that may permit the mechanical attachment of one piece to another, and further includes bifurcated stent grafts having a support structure for loading the stent graft into a catheter with less bunching of the graft material than in conventional devices.

### FIELD OF THE INVENTION

The present invention relates to intravascular devices, and more particularly, to bifurcated stent grafts, and apparatus and methods for making same.

### BACKGROUND OF THE INVENTION

The present invention relates to a bifurcated stent graft and apparatus and methods for making same. The stent graft is suited for delivery into an organ, such as one or more arteries, of a human or animal. As used herein, the following terms shall have the definitions hereafter ascribed: “Lumen” refers to a channel or cavity within an organ, such as an artery or other blood vessel. Each of the terms “intravascular device,” “endoluminal device,” “intraluminal device” and “endoprosthesis” means a device designed for placement inside one or more lumens in the body, e.g., in the lumen of a blood vessel, and such device can be a stent, stent graft, occluder or other device. “Transluminal device” or “transluminal structure” refers to a device that connects the lumen of one organ, such as a blood vessel, to the lumen of another organ, such as another blood vessel. The term “occlude” means closed off or blocked, and an “occluder” is a device designed to close off or block a lumen. The term “blood vessel” refers to a structure, such as an artery or vein, which transports blood. “Catheter” means a generally tubular, surgical instrument inserted into a body cavity, such as into a lumen of a blood vessel.

A “stent” is a device that provides support to, and/or expands, a weakened or clogged vessel in order to maintain an opening for sufficient fluid (particularly blood) flow. Stents are often placed in blood vessels and apply outward pressure against the blood vessel wall. Stents may be used to repair compromised coronary arteries that have become narrowed (called a stenosis) or blocked by the build up of plaque. Stents may also be used to support structures that are being anastomosed.

A stent covered or lined with a biocompatible material to form a channel through which blood or other fluid flows is known as a stent graft, prosthetic vascular graft or endoluminal graft. Known stent grafts are generally tubular structures (either right-cylindrical tubes or other shapes, such as conical) that allow for the passage of fluid, such as blood, therethrough. For example, stent grafts formed of biocompatible materials (e.g., Dacron or expanded, porous polytetrafluoroethylene (ePTFE) tubing) have been employed to replace or bypass occluded or damaged blood vessels. They may also be used to repair or replace weakened or diseased blood vessels, such as the aorta, which have developed dilated (or enlarged), weakened areas known as aneurysms. In the aorta, aneurysms may often occur near an area where the aorta connects with secondary arteries, such as the two common iliac arteries, which supply blood to the lower limbs. Stent grafts are disposed in the artery and bridge the aneurysm in order to remove the pressure on the weakened artery wall and reduce the risk of rupture (called an embolism), which is when the natural wall bursts. The use of stents and stent grafts for treatment or isolation of vascular aneurysms and vessel walls which have been thinned or thickened by disease (this use is called endoluminal repair or exclusion) is thus known.

There are many types of stents and stent grafts. A vascular stent that comprises a length of sinuous or “zigzag” wire formed into a helix or sinuous configuration is disclosed in U.S. Pat. No. 4,886,062. The stent defines a generally cylindrical structure which, in use, constitutes a prosthetic intraluminal wall. The sinuous configuration of the wire permits radial compression and expansion of the stent. The patent discloses that the stent can be delivered percutaneously and expanded in situ using a balloon catheter. This same general stent configuration may also be used in stent grafts. Some stents are generally flexible so they can be easily maneuvered through the various body vessels for deployment. Once in position, the stent may be deployed by allowing the stent to expand to its uncompressed state or by expanding the stent by use of a catheter balloon.

There are also “self-expanding” stents and stent grafts, i.e., which are inserted into the vascular system in a compressed or contracted state, and self-expand upon removal of a restraint, rather than having to be expanded using a balloon. Some self-expanding stents and stent grafts employ a wire of suitable material, such as stainless steel or nitinol (nickel-titanium), configured to provide an outward radial force. Such a wire is typically formed to be substantially circular and had a slightly greater diameter than the diameter of the lumen in which it is intended to be used.

The use of trigger or release wires to control expansion of a self-expanding endoprosthesis are known. Additionally, U.S. Pat. No. 5,415,664 describes a stent delivery and deployment apparatus including three concentric tubes, an interior hollow tube and an outer sheath, and an inner tubular actuation piece with a cup-like gripping piece rigidly attached to its distal end. Other devices for deploying self-expanding endoprosthesis are described in U.S. Pat. Nos. 5,484,444, 5,833, 5,776,142, 5,873,906, and 5,700,269.

An expandable intraluminal stent graft which is constituted by a tubular piece formed from a plurality of intersecting elongate pieces which permit radial expansion and compression of the stent is disclosed in U.S. Pat. No. 4,733,665.

An intraluminal stent which is constituted by a sinuous wire formed into a helix is disclosed in EP-A-0556850. Juxtaposed apices of the wire are secured to one another so that each hoop of the helix is supported by its neighboring hoops to increase the overall strength of the stent and to minimize the risk of plaque herniation. In some embodiments the stent of EP-A-0556850 further comprises a tubular graft piece to form an endoluminal prosthesis.

Intravascular devices, such as stents, stent grafts and occluders, are deployed in the body using different methods. They may be deployed using a “cut-down” procedure, i.e., cutting directly into the lumen from an entry point proximate to the site where the prosthesis is to be deployed and placing the device into the lumen. Alternatively, they may be deployed using a less invasive percutaneous method, such as cutting through the skin to access a lumen at a convenient, and relatively low-trauma, entry point, and routing the device through the lumen until the site where the device is to be deployed is reached. Deployment of an intravascular device is sometimes effected using a delivery catheter with coaxial inner (plunger) and outer (sheath) tubes arranged for relative axial movement. The device is compressed and disposed within the distal end of the outer catheter tube in front of the inner tube. The distal end of the catheter is then maneuvered, typically routed though a lumen, until it (and thus the intravascular device) is positioned in the vicinity of the intended treatment site. The inner tube is then held stationary when the outer tube of the delivery catheter is withdrawn. The inner tube prevents the intravascular device from being withdrawn with the outer tube, so that, as the outer tube is withdrawn, the intravascular device radially expands into a substantially conforming surface contact with the interior of the lumen. An example of such a delivery system is described in U.S. Pat. No. 4,655,771, the disclosure of which is incorporated herein by reference.

It is also known to insert and advance an intravascular device in the form of a unitary bifurcated graft through a single branch of the femoral arterial system, to a point beyond the treatment site, then pull or draw one of the limbs into the contralateral (opposite) branch by manipulation of a contralateral-femoral wire catheter or snare. Such a system is described in U.S. Pat. No. 5,639,278, the disclosure of which is incorporated herein by reference. An example of another deployment system is one that requires cross-femoral wire catheter and guide wires, and is described in U.S. Pat. No. 5,489,295 and PCT Application No. WO 98/36708. It is also suggested in U.S. Pat. No. 4,617,932 that blood flow entering the graft can be utilized to cause the graft to float free in the blood stream so that it may be directed to the proper position.

The application of bifurcated stent grafts to branched lumen (such as the infrarenal portion of the aortic artery where it bifurcates to the common iliac arteries) is also known. However, the deployment of a bifurcated stent graft is typically relatively invasive because the respective portions of bifurcated stent grafts often must be joined in situ and require a plurality of catheterizations. Stent grafts for bifurcated lumen are described in U.S. Pat. Nos. 5,906,640, 5,755,734, and 5,827,320. These devices require large access ports in a portion of the vessel that is usually much deeper under the skin than the preferred femoral artery entry site.

The respective disclosures of the following U.S. Patents are also incorporated herein by reference to describe the general state of the art: U.S. Pat. No. 6,183,481 entitled “Delivery System for Self-expanding Stents and Grafts,” filed on Sep. 22, 1999; U.S. Pat. No. 6,402,779 entitled “Balloon-assisted Intraluminal Stent Graft,” filed on Jul. 26, 1999; U.S. Pat. No. 6,443,981 entitled “Expandable Vascular Prosthesis” filed on May 19, 2000; U.S. Pat. No. 6,187,054 entitled “Method of Making Large Diameter Vascular Prosthesis Made by Said Method,” filed on Feb. 4, 1999, and U.S. Pat. No. 6,605,119 entitled “of Making Large Diameter Vascular Prosthesis Made by Said Method,” filed on Nov. 1, 2000. Other various stents and/or stent grafts are described in U.S. Pat. Nos. 5,873,906, 5,718,724, 5,302,317, 5,662,713, 5,575,816, 5,0507,767, 5,415,664, 4,955,899, 4,800,882, 4,907,336, and 5,152,782.

The prior art intravascular devices are generally satisfactory for the treatment of aneurysms, stenoses and other angeological diseases at sites in blood vessels. The prior art does not disclose, however, tools for efficiently forming bifurcated stent grafts or the stent grafts made by such tools. Moreover, the prior art does not disclose stent grafts that may be safely and securely attached to one another to prevent, for example, the inadvertent separation of one from the other. The prior art also fails to disclose bifurcated stent grafts that may be loaded into a catheter without bunching of the device near the bifurcation, which can make loading or deployment difficult. Such bunching may also have the undesirable effect of delaminating one or both of the two layers of biocompatible material, either from the support or each other, while the stent graft is loaded into a catheter.

### SUMMARY OF THE INVENTION

The present invention solves the above-referenced problems. In one embodiment, a tool is disclosed for forming an intravascular device such as a bifurcated stent graft. The tool comprises a plurality of tool pieces, each piece preferably has at least a portion that is tubular in shape, wherein at least one tool piece is removably coupled with another tool piece to facilitate simple manufacture of the tool and/or easy removal of the endovascular device from the tool, and the external surface of the tool is used to assist in forming the intravascular device.

In another embodiment, a method is disclosed for forming a bifurcated stent graft. The method comprises applying a first layer of biocompatible material over an external surface of a tool for forming the stent graft, applying a support structure over the first layer, and applying a second layer of biocompatible material over the support structure and the first layer.

In another embodiment, a two-piece stent graft is disclosed, which comprises a first stent graft and a second stent graft. The first graft comprises at least one pocket or cavity, and the second graft comprises at least one projection, wherein the projection is preferably a stent having a portion protruding from the second graft. The projection is received in the pocket to assist in attaching the first graft to the second graft.

In another embodiment, a stent graft is disclosed comprising a first layer of biocompatible material, a support structure over the first layer, a second layer of biocompatible material over the support structure and the first layer, and at least one pocket formed between the first layer and the second layer, the pocket for receiving a projection.

In another embodiment, a stent graft is disclosed comprising a first layer of biocompatible material, a support structure over the first layer, a second layer of biocompatible material over the support structure and the first layer, and at least one projection, which is preferably a portion of the support structure, protruding from the stent graft.

In another embodiment, a bifurcated stent graft is disclosed comprising a first layer of biocompatible material, a support structure over the first layer and including a portion that extends at least partially into a bifurcated section in the stent graft, and a second layer of biocompatible material over the support structure and the first layer. The portion of the support structure extending into the bifurcated section assists in loading the stent graft into a catheter without bunching the biocompatible material or separating the layers of biocompatible material.

Advantages of the invention will be set forth in part in the description which follows, and in part will be obvious from the description, or may be learned by practice of the invention. The advantages of the invention will be realized and attained by means of the elements and combinations particularly pointed out in the appended claims.

Neither the foregoing general description nor the following detailed description are exemplary and explanatory only and are not restrictive of the invention as claimed.

The accompanying drawings, which are incorporated in and constitute a part of this specification, illustrate several embodiments of the invention and together with the description, serve to explain the principles of the invention.

### BRIEF DESCRIPTION OF THE DRAWING

FIGS. 1A-1D show, in progressive stages of assembly, apparatus for forming intravascular devices, in accordance with systems and methods consistent with the present invention.

FIG. 2 is a flowchart depicting a method for forming intravascular devices, in accordance with systems and methods consistent with the present invention.

FIGS. 3A-3F depict forming an intravascular device, in accordance with systems and methods consistent with the present invention.

FIG. 3G is a close-up perspective view of part of the intravascular device of FIG. 3F, shown without the forming tool and with part of the device broken away, in accordance with systems and methods consistent with the present invention.

FIG. 3H is a close-up perspective view of part of the intravascular device of FIG. 3F, shown without the forming tool, in accordance with systems and methods consistent with the present invention.

FIG. 4 is a close-up perspective view of part of an intravascular device, in accordance with systems and methods consistent with the present invention.

### DESCRIPTION OF THE PREFERRED EMBODIMENTS

Reference will now be made in detail to the present exemplary embodiments of the invention, examples of which are illustrated in the accompanying drawings. Wherever possible, the same reference numbers will be used throughout the drawings to refer to the same or like parts.

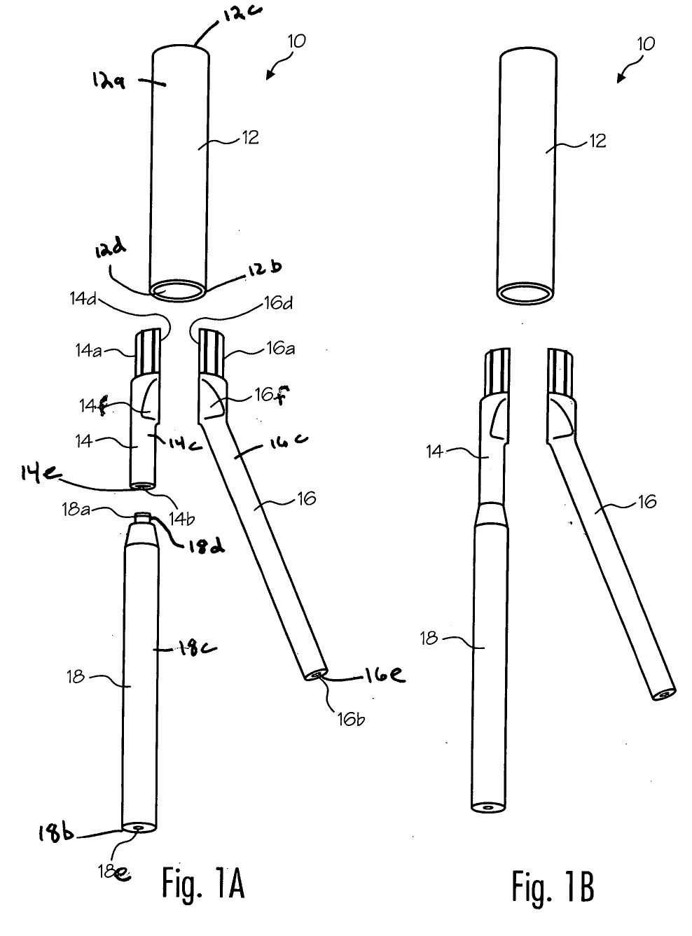

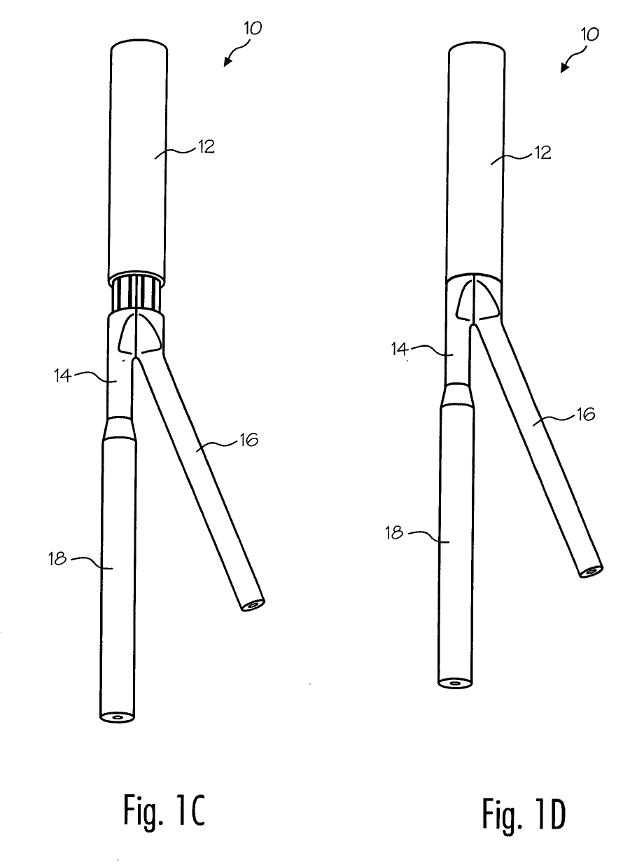

FIG. 1A shows a tool 10 in disassembled (or decoupled) form, wherein the tool is for forming an intravascular device, such as a bifurcated stent graft. Tool 10 as shown is for forming a bifurcated stent graft having a portion to be received in the aorta and two portions to be received, respectively, in each of the iliac arteries. However, a tool according to the invention could be used for forming any type of bifurcated intravascular device. FIG. 1B shows piece 18 coupled to piece 14; FIG. 1C shows pieces 14 and 16 being coupled to piece 12 and FIG. 1D shows tool 10 fully assembled.

Tool 10 includes a plurality of tool pieces: aorta tool piece 12, first iliac tool piece, which is preferably a two-part structure comprising a proximal aorta section 14 and a distal aorta section 18, and a second iliac tool piece 16. When assembled, tool 10 provides an external surface for forming an intravascular device, as best seen in Fid. 1D. The size and shape of tool pieces comprising tool 10 may be selected to provide any desired external surface for assisting in forming an intravascular device with a corresponding size and shape. Additionally, the pieces of tool 10 may be made of any suitable material, such as 6061-T6 aluminum having a polished exterior surface, having the desired properties for forming intravascular devices according to the invention.

Piece 12 is for forming the portion of the bifurcated stent graft that will be positioned in the aorta. Piece 12 is preferably tubular, hollow and preferably has a generally smooth, cylindrical external surface 12a, a first end 12b, a second end 12c and an inner cavity 12d. Piece 12 may include on the surface of the wall defining cavity 12d any structure suitable to facilitate alignment with and/or removable coupling with piece 14 and piece 16, such as ribs (not shown).

Piece 14 is for assisting in forming a portion of the bifurcated stent graft that will be positioned in one of the iliac arteries. Proximal aorta section 14 preferably includes a first end 14a, a second end 14b, an exterior surface 14c, a connector 14d, and a bore 14e. Connector 14d is positioned at first end 14a and is any structure suitable for aligning section 14 with and/or removably coupling section 14 with piece 12. As shown, connector 14d has a semi-circular cross section with a flat side and grooves. Section 14 preferably includes a bore 14e on second end 14b, but bore 14e may be any suitable structure for coupling proximal aorta section 14 to distal aorta section 18.

Distal aorta section 18 has a first end 18a, a second end 18b, an exterior surface 18c, and a pin 18d. As shown the diameter of distal aorta section 18 is greater than the diameter of proximal aorta section 14, and in this embodiment exterior surface 18c generally defines the dimensions of the portion of the stent graft formed on section 18. First end 18a is conical and a pin 18d (preferably made of steel) extends therefrom. Pin 18d is received in bore 14e to removably couple section 18 to section 14 and any structure suitable for this purpose may be used. A bore 18e is formed in end 18b. In this embodiment, the purpose for removably coupling section 18 to section 14 is for easy removal of the bifurcated stent graft from tool 10 after being formed.

Piece 16 is for assisting in forming a portion of the bifurcated stent graft that will be positioned in the iliac artery opposite the stent graft formed on piece 14. Piece 16 includes a first end 16a, a second end 16b, and an exterior surface 16c. A connector 16d is generally semi-circular in shape and includes grooves for assisting in aligning and/or removably coupling piece 16 with piece 12, and any suitable structure for performing this function may be used. Second end 16b includes a bore 16e. Piece 16 includes a generally flat surface 16f that aligns with another generally flat surface 14f on piece 14 when tool 10 is assembled.

The tool pieces can be coupled together in any order. It is preferred that pieces 14 and 16 first be pressed together and then be inserted into cavity 12d.

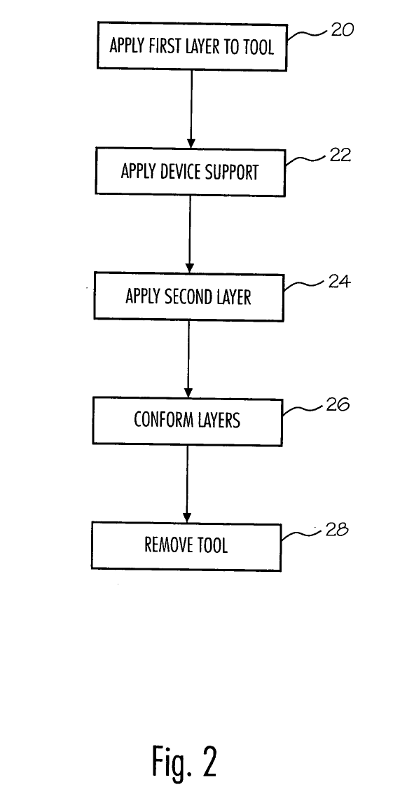

FIG. 2 is a flowchart depicting a method for forming an intravascular device with a tool, such as tool 10 in FIG. 1D. Those skilled in the art will appreciate, however, that another tool could be utilized to perform this method.

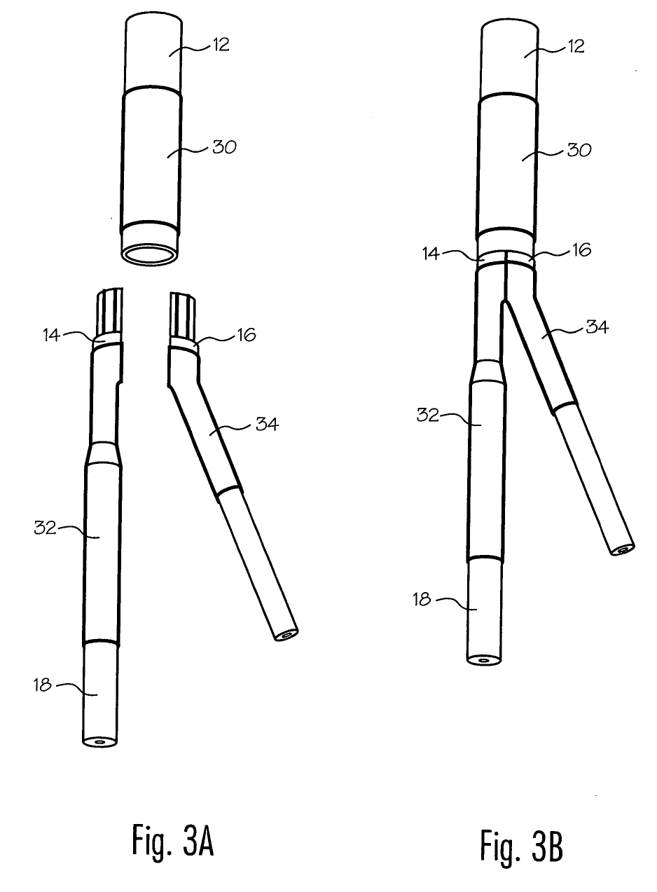

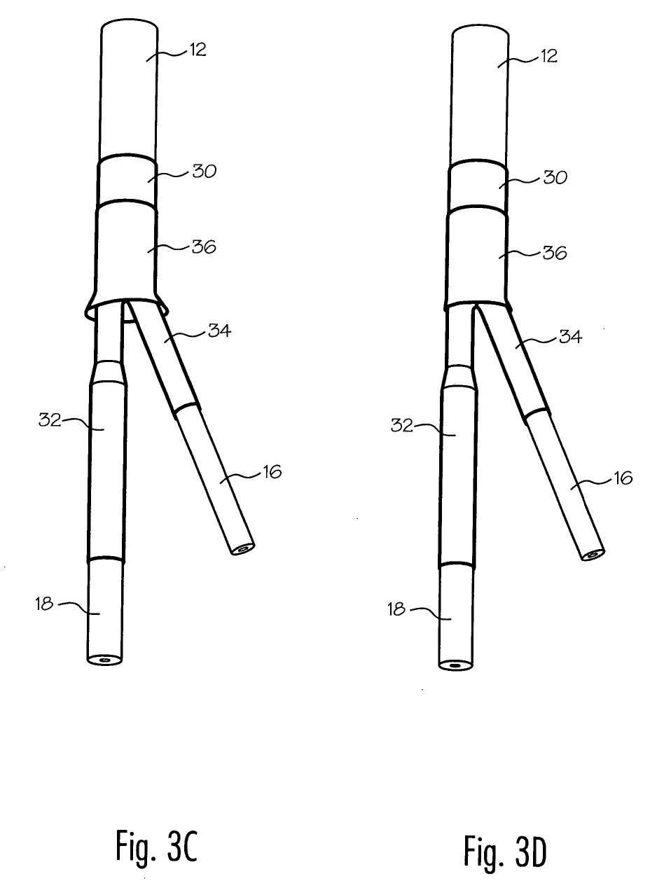

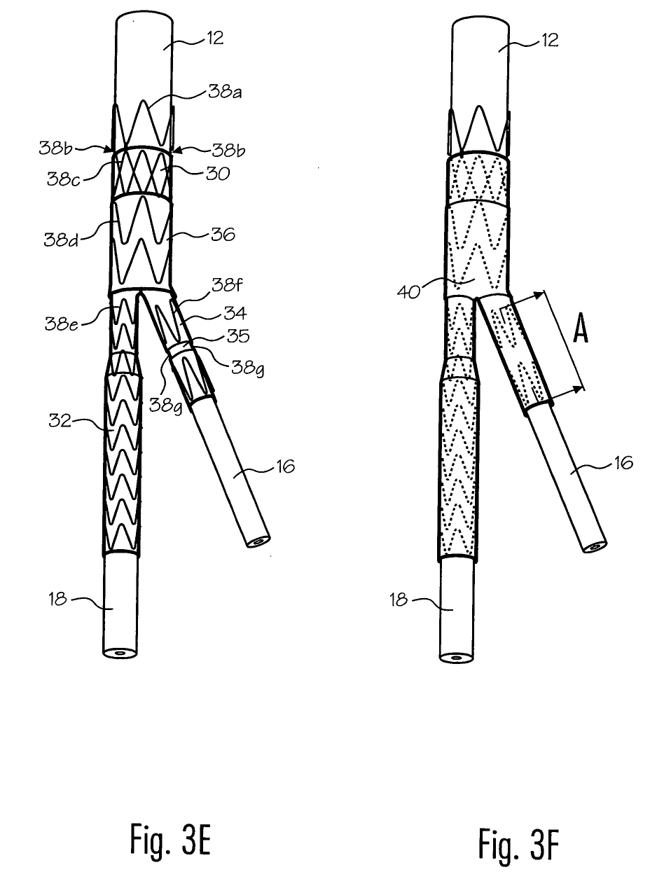

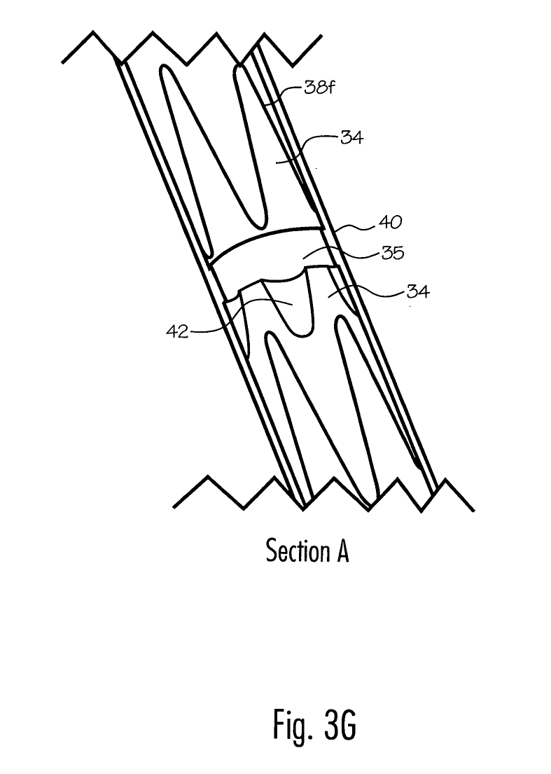

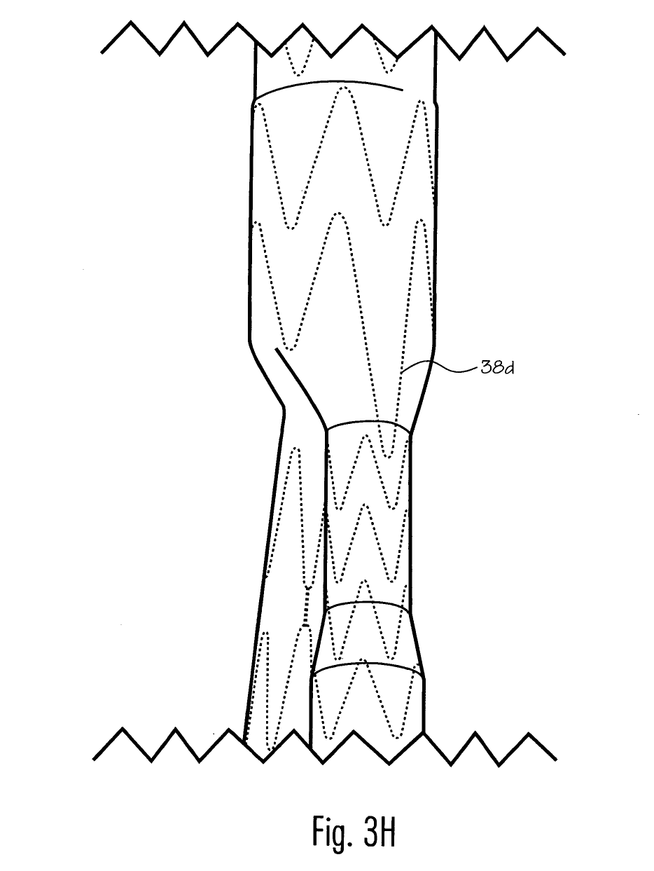

In step 20, a layer of biocompatible material is applied over tool 10. The material may comprise any desired biocompatible material, such as porous polytetrafluoroethylene (ePTFE). Moreover, the layer need not (and preferably does not) cover the entire external surface of assembled tool 10, although it may. In the preferred method step 20 comprises placing a first layer of biocompatible material over tool piece 12, another first layer of biocompatible material over tool piece 14 and 18 and another first layer of biocompatible material over tool piece 16. FIGS. 3A-3D depict an example process for applying a layer of biocompatible material over tool 10.

FIG. 3A shows tool 10 in partially assembled form with layers of biocompatible material placed on pieces 12,14, 18 and 16, wherein the layers are preformed tubes 30, 32 and 34, respectively. Any suitable methods for manufacturing the tubes of biocompatible material may be utilized, such as the methods disclosed in some of the aforementioned U.S. patents incorporated herein by reference. Unlike the devices depicted in FIG. 3A, which shows tubes 32 and 34 snuggly conforming to the external surfaces of the respective tool pieces upon which they are mounted, tubes 32 and 34 may not initially so conform. However, those skilled in the art appreciate that tubes 30, 32 and 34 may be made to any desired size and shape satisfactory to fit over the corresponding pieces of tool 10.

FIG. 3B shows tool 10 in assembled form with pieces 30, 32 and 34 mounted thereon. Some regions of tool 10 may not, as yet, be covered with biocompatible material. For example, the region of tool 10 between tubes 30, 32 and 34 may not be covered, but as shown in FIG. 3C, biocompatible material 36 may be applied to cover this region. Biocompatible material 36 may be applied as a tube that is slip-fit over the exposed region, a tape that is laid over the exposed region, or any other appropriate material.

FIG. 3D shows biocompatible material 36 snuggly conforming to tool 10, which may be accomplished in any suitable manner, such as by adjusting the tubes, trimming excess material, and/or heat shrinking (preferred) by placing tool 10 with the first biocompatible layers into an oven. Such heat shrinking may be performed by covering tool 10 and the layer of biocompatible material with a heat shrinking material, such as a plastic film, and preferably heating it in a convection oven for any desired period, for example, at about 250-280 degrees Celsius for about 2-5 minutes.

In step 22 of FIG. 2, a support structure is applied over the first layer of biocompatible material overlaying tool 10. The support structure may comprise any desired support structure for intravascular devices, such as one or more stents. Any suitable number and layout of support structures, such as stent(s), for a particular stent graft may be utilized, as those skilled in the art understand. The preferred stent grafts are formed of nitrile wire and have a sinusoidal shape as shown.

FIG. 3E depicts an example support structure that is applied over the layer of biocompatible material on tool 10. In FIG. 3E, the support structure includes a plurality of stents 38a, 38c, 38d, 38e and 38f, each of which may be conventionally applied in any desired patterns, such as sinusoid patterns of differing amplitude, as shown by way of example. When utilized, stent 38a may be attached by one or more connectors 38b to stent 38c. As shown, stent 38a may extend above the layer of biocompatible material. Stent 38c may include more than one stent, overlaying each other as shown. Stents 38f may be connected by one or more connectors 38g to provide more stability to the overall stent graft. A portion 35, such as a strip, may be removed from the biocompatible material layer overlaying tool 10.

In step 24 of FIG. 2, a second layer of material, such as biocompatible material, may be applied over the support structure and the first layer applied in step 20. The second layer applied in step 24 may be applied as described for the first layer, i.e., preformed tubes of material such as ePTFE, or in any other suitable manner. FIG. 3F depicts layer 40 which has been applied over the support structure and the first layer applied in step 20. To clarify, FIG. 3F shows the applied layers as snuggly conforming to tool 10, however, in practice a conforming process may first have to be performed to reduce the size of layer 40 and make it fit snuggly to tool 10. In this embodiment layer 40 is also comprised of multiple tubes that are applied to tool 10.

In step 26, a process may be performed to conform the applied layers as desired to the external surface of tool 10. For example, such conforming may involve covering tool 10 and the layers of biocompatible material with a heat shrinking material, such as a plastic film, and heating, preferably in a convection oven, for any desired period, for example, at about 355-365 degrees Celsius for about 17-18 minutes.

In step 28, after allowing tool 10 and the device to cool as desired, tool 10 and the device are separated. Pieces 12, 14, 16 and 18 may be separated from each other using their respective removable connectors, thus leaving the intravascular device, which may then be further trimmed to desired shape.

Those skilled in the art appreciate that the above-described manner of applying layers of biocompatible material is exemplary only, understanding that presently well-known or future alternatives for applying layers or support structures may be employed. For example, one may envision applying such layers by employing one or more sheets, as opposed to tubes, or by spraying or otherwise depositing layers of desired materials.

Referring to FIG. 3G, a close-up perspective view is shown of a region “A” of the intravascular device of FIG. 3F, however, for drawing simplification, FIG. 3G does not show tool 10 and portions of the outer layer of biocompatible material are removed. FIG. 3G shows a plurality of pockets 42 that may be formed on an interior surface of the device, between the layers of the device. Pockets 42 may be formed by separating the device layers, as desired. For example, after the device is removed from tool 10, one may use an instrument to reach into the device and separate the layers as desired. Removing a portion 35 of the layer from step 20 may facilitate this process. Those skilled in the art will appreciate, however, that any suitable manner of pocket formation may be utilized.

FIG. 3H is a close-up perspective view of part of the intravascular device of FIG. 3F, shown without tool 10. As shown, a support structure, such as stent 38d, may include a portion that extends at least partially across a bifurcation or branch in the device. Such support structure may extend across the bifurcation a greater or lesser degree than shown. The added support limits bunching of the device near the bifurcation region that may otherwise occur during device loading into a catheter.

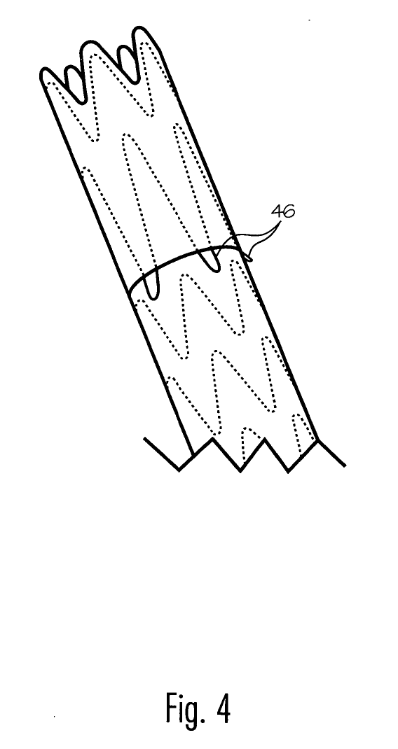

FIG. 4 is a close-up perspective view of part of an intravascular device that may be made using tools and methods disclosed herein, or using any other tool or method. At least one portion of the support structure, such as a stent 46, has been exposed. This may be done by, for example, conventionally delaminating the outer layer of biocompatible material in proximity to the desired support structure portions. Such extended support structure portions may be employed to attach the device of FIG. 4 to, for example, the device of FIG. 3G. Such attachment may be accomplished by, for example, inserting the device of FIG. 4 (in a catheter) into an already-deployed device of FIG. 3G and deploying and withdrawing the former device until the protruding support structure portion(s) catch respective pocket(s) in the latter device.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims and legal equivalents thereof.

1. A tool for forming an intravascular device, the tool comprising:
a plurality of tool pieces removably coupled together; and
an external surface for assisting in forming the intravascular device, wherein the intravascular device is formed at least in part on the external surface of the tool.

2. The tool of claim 1 wherein at least one of the tool pieces has a cylindrical external surface.

3. The tool of claim 1 wherein at least one of the tool pieces is hollow.

4. The apparatus of claim 1 where at least one tool piece comprises aluminum.

5. The tool of claim 1 wherein the tool comprises at least three tool pieces including an aorta piece, a first iliac piece and a second iliac piece.

6. The tool of claim 1 wherein the aorta piece has a diameter, the first iliac piece has a diameter and the second iliac piece has a diameter, the diameter of the aorta piece being greater than the diameter of the first iliac piece and greater than the diameter of the second iliac piece.

7. The tool of claim 5 wherein there are at least four tool pieces.

8. The tool of claim 5 wherein the first iliac piece and second iliac piece each include a connector and the aorta piece includes a cavity and each of the connectors is received in the cavity when the tool pieces are coupled together.

9. The tool of claim 7 wherein the first iliac piece has two sections and comprises a proximal aorta section and a distal aorta section.

10. The tool of claim 9 wherein the proximal aorta section is removably connected to the distal aorta section.

11. The tool of claim 10 wherein the proximal aorta section has a bore and the distal aorta section has a pin, the proximal aorta section being removably connected to the distal aorta section when the pin is received in the bore.

12. The tool of claim 9 wherein the proximal aorta section has a diameter and the distal aorta section has a diameter, the diameter of the distal aorta section being larger than the diameter of the distal aorta section.

13. A method for forming a stent graft, the method comprising:
applying a first layer of biocompatible material to an external surface of a tool for forming the stent graft;
applying a support structure over the first layer; and
applying a second layer of biocompatible material over the support structure.

14. The method of claim 13 further comprising removing a portion of the first layer.

15. The method of claim 14 further comprising forming at least one pocket between the first layer and the second layer along an edge of the first layer where the portion was removed.

16. The method of claim 13 wherein the support structure comprises one or more stents.

17. The method of claim 16 wherein at least one of the one or more stents has a sinusoid pattern.

18. The method of claim 13 further comprising heating the layers.

19. The method of claim 13 wherein the tool comprises a plurality of tool pieces removably coupled together.

20. The method of claim 19 further comprising the steps of decoupling at least one of the tool pieces from another of the tool pieces and removing the stent graft from the tool.

21. The method of claim 13 wherein the first layer is comprised of ePTFE.

22. The method of claim 13 wherein the second layer is comprised of ePTFE.

23. The method of claim 17 wherein the one or more stents are comprised of nitrile wire.

24. The method of claim 13 wherein the stent graft is a bifurcated stent graft.

25. The method of claim 24 wherein the tool comprises at least three tool pieces, an aorta piece, a first iliac piece and a second iliac piece.

26. The method of claim 25 wherein the bifurcated stent graft comprises an aorta stent graft section formed on the aorta piece, first iliac stent graft formed on the iliac piece and a second iliac stent graft formed on the second iliac piece, the aorta stent graft, the first iliac stent graft and the second iliac stent graft being bonded together.

27. The method of claim 25 wherein the aorta stent graft includes a second layer of biocompatible material that overlaps the first iliac stent graft and the second iliac stent graft to assist in bonding together the aorta stent graft, the first iliac stent graft and the second iliac stent graft.

28. The method of claim 25 wherein adhesive is used to assist in bonding together the aorta stent graft, the first iliac stent graft and the second iliac stent graft.

29. The method of claim 24 wherein the first iliac piece comprises a proximal aorta section and a distal aorta section, the proximal aorta section and distal aorta section being removably coupled together.

30. The method of claim 29 wherein the bifurcated stent graft is removed from the tool when the proximal aorta section and distal aorta section are not coupled together.

31. The method of claim 13 that further includes heating the stent graft.

32. The method of claim 31 that further includes wrapping the stent graft with plastic sheet material before heating it.

33. The method of claim 31 wherein the stent graft is on the tool when heated.

34. The method of claim 31 wherein the stent graft is heated for 2-5 minutes at 280-300 degrees Celsius.

35. An apparatus for attaching an intravascular device to a second intravascular device, the apparatus comprising:
at least one pocket in the first device; and
at least one projection protruding from the second device and for being received in the at least one pocket.

36. The apparatus of claim 35 wherein the at least one pocket opens on an internal surface of the first device.

37. The apparatus of claim 35 wherein the projection is a portion of a stent that protrudes from the second device.

38. The apparatus of claim 37 wherein the portion protrudes outwardly from an external surface of the second device.

39. The apparatus of claim 35 wherein the at least one pocket is formed between a first layer of biocompatible material of the first device and a second layer of biocompatible material of the first device.

40. The apparatus of claim 39 wherein the biocompatible material is ePTFE.

41. The apparatus of claim 39 wherein the first intravascular device is a stent graft and the second intravascular device is a stent graft.

42. The apparatus of claim 41 wherein the first intravascular device is a bifurcated stent graft.

43. A first stent graft comprising a pocket to receive a projection of a second stent graft in order to connect the first stent graft to the second stent graft.

44. The first stent graft of claim 43 comprising:
a first layer of biocompatible material;
a support structure over the first layer;
a second layer of biocompatible material over the support structure and the first layer; and
a pocket formed between the first and second layers

45. A first stent graft comprising a projection for being received in a pocket of a second stent graft in order to connect the first stent graft to the second stent graft.

46. The stent graft of claim 45 that further comprises:
a first layer of biocompatible material;
a support structure over the first layer;
a second layer of biocompatible material over the support structure and the first layer; and
at least one aperture in the second layer through which a portion of the support structure protrudes, wherein the protruding part of the support structure is the projection.

47. An intravascular device, comprising:
a first layer of biocompatible material;
a support structure over the first layer and including a portion that extends at least partially across a bifurcated portion of the device; and
a second layer of biocompatible material over the support structure and the first layer.

48. The device of claim 47 wherein the support structure comprises at least one stent.

49. The device of claim 48 wherein the at least one stent comprises a stent having a sinusoid pattern.

50. The device of claim 49 wherein the sinusoid pattern of the stents includes more than one amplitude.

51. The device of claim 50 wherein the portion that extends at least partially into the bifurcated section is an apex of a sinusoid wave of one of the at least one stent grafts.
